(19) United States
(12) Patent Application Publication
(10) Pub. No.: US 2024/0327793 A1
(43) Pub. Date: Oct. 3, 2024

(54) CULTURED ADIPOSE TISSUE

(71) Applicant: Trustees of Tufts College, Medford, MA (US)

(72) Inventors: David Kaplan, Medford, MA (US); John Se Kit Yuen, Medford, MA (US)

(21) Appl. No.: 18/294,033

(22) PCT Filed: Aug. 5, 2022
(86) PCT No.: PCT/US22/74641

§ 371 Date: Jan. 31, 2024

Related U.S. Application Data
(60) Provisional application No. 63/203,980, filed on Aug. 5, 2021.

(51) Int. Cl. C12N 5/077; C12M 1/12; C12M 3/06

(52) U.S. Cl. CPC C12N 5/0653; C12M 25/10; C12M 27/14; C12N 2501/72; C12N 2506/1384; C12N 2513/00; C12N 2533/74; C12N 2537/10

(57) ABSTRACT

The present disclosure relates to cell cultured adipose tissue. In one embodiment, the cultured adipose tissue is produced by culturing adipose cells in a culture media in vitro, harvesting the adipose cells after a desired amount of adipose cells are produced, and aggregating the harvested adipose cells to provide the cultured adipose tissue. In some embodiments, aggregating the harvested adipose cells comprises mixing the harvested adipose cells with a hydrogel or binder in a three-dimensional (3D) mold. In other embodiments, aggregating the harvested adipose cells comprises cross-linking the harvested adipose cells in a 3D mold. The cultured adipose tissue have a defined 3D shape and a size on the macroscale. In some embodiments, the cultured adipose tissue may be a food product.

### CROSS-REFERENCE TO RELATED APPLICATION

This application is related to, claims priority to, and incorporates herein by reference for all purposes U.S. Provisional Patent Application No. 63/203,980, filed Aug. 5, 2021.

### FIELD OF INVENTION

Conventional animal agriculture for the production of meat (muscle and fat tissue) is linked to numerous drawbacks such as environmental degradation, zoonic disease emergence, antimicrobial resistance, and animal welfare concerns. To provide the world with alternatives to animal products having reduced negative impacts on animals and the environment, there is increasing interest in producing cultured (in vitro) fat tissue.

### BACKGROUND

Conventional animal agriculture for the production of meat (muscle and fat tissue) is linked to numerous drawbacks such as environmental degradation, zoonic disease emergence, antimicrobial resistance, and animal welfare concerns. To provide the world with alternatives to animal products having reduced negative impacts on animals and the environment, there is increasing interest in producing cultured (in vitro) fat tissue.

In the field of alternative proteins, existing solutions to recapitulate the fat content of meat largely revolve around the direct addition or utilization of plant fats and oils (e.g., coconut oil). Recent developments in this realm include the use of oleogels, where nanoscale globules of vegetable oils are generated to better create the texture of solid fats (i.e., animal fats). However, plant-based fats do not incorporate the often complex aroma and flavor of meat, as well as species-specific flavors that distinguish meat from cows, pigs, chicken, fish, and so on.

Native (in vivo) adipose tissue is largely a dense packing (aggregation) of lipid-filled adipocytes held together by a sparse extracellular matrix (ECM). This is opposed to muscle tissue which is comprised of aligned fibers in a multi-hierarchical structure. To date, three-dimensional (3D) culture has been the main approach for generating bulk/macroscale tissues. These tissue engineering strategies involve the in vitro growth of cells over 3D scaffolds. However, it is challenging to scale up 3D culture due to mass transport limitations with regard to oxygen, nutrients, and waste. It is often quoted in the field that cells cannot remain viable in 3D tissues unless they are within about 200 micrometers of a source of blood or culture media. Overcoming these challenges to maintain cell viability in 3D tissues may require vascularization or the incorporation of an elaborate tissue perfusion system to distribute nutrients to the cells. It is currently infeasible to directly grow large tissues on the macroscale (millimeter scale and up) using contemporary tissue engineering techniques without the use of perfusion or related methods or with the structural features of fat as found in vivo. Likely due to these challenges, large-scale production of adipose tissue that mimics those found in vivo does not appear to have been implemented to date.

Thus, there remains a need for strategies for the large-scale production of cultured adipose tissue. The present disclosure provides a technical solution to this need.

### SUMMARY

Disclosed herein is a method for producing cultured adipose tissue. The method may include growing adipogenic precursor cells in a first culture media, differentiating the adipogenic precursor cells to adipose cells in a second culture media, and harvesting the adipose cells. The method may further include aggregating the harvested adipose cells to provide the cultured adipose tissue. In some embodiments, growing the adipogenic precursor cells and differentiating the adipogenic precursor cells to adipose cells is carried out in a bioreactor.

Further disclosed herein is a method for producing cultured adipose tissue. The method may include growing adipogenic precursor cells in a culture media, and differentiating the adipogenic precursor cells to adipose cells in the culture media. The method may further include harvesting the adiposed cells, and aggregating the harvested adipose cells to provide the cultured adipose tissue.

Further disclosed herein is a method for producing cultured adipose tissue. The method may include growing adipogenic precursor cells on a two-dimensional (2D) substrate, differentiating the adipogenic precursor cells to adipose cells on the 2D substrate, and harvesting the adipose cells. The method may further include aggregating the harvested adipose cells to provide the cultured adipose tissue. In some embodiments, the 2D substrate is a conveyor belt and the method is carried out in a continuous, assembly line-like process.

Also disclosed herein is a method for producing cultured adipose tissue. The method may include culturing adipose cells from adipogenic precursor cells in culture media, harvesting the adipose cells after a desired amount of adipose cells are produced, and aggregating the harvested adipose cells to provide the cultured adipose tissue.

Further disclosed herein is cultured adipose tissue. The cultured adipose tissue may include adipose cells embedded in a hydrogel or binder. The cultured adipose tissue may have a 3D shape and a size on the macroscale. In some embodiments, the cultured adipose tissue is a food product.

Further disclosed herein is cultured adipose tissue. The cultured adipose tissue may include adipose cells cross-linked together. The cultured adipose tissue may have a 3D shape and a size on the macroscale. In some embodiments, the cultured adipose tissue is a food product.

### BRIEF DESCRIPTION OF THE DRAWINGS

FIG. 1 is a schematic representation of cultured adipose tissue, in accordance with the present disclosure.

FIG. 2 is a flow chart of steps that may be involved in producing the cultured adipose tissue, in accordance with the present disclosure.

FIG. 3 is a schematic representation of methods of producing the cultured adipose tissue using bioreactors, in accordance with the present disclosure.

FIG. 4 is a schematic representation of a continuous process of producing the cultured adipose tissue on a conveyor belt, in accordance with the present disclosure.

FIG. 5 is a timeline of 3T3-L1 adipogenic differentiation, according to an embodiment of the present disclosure.

FIG. 6 is a schematic representation of methods of producing the cultured adipose tissue using a rotating wall vessel bioreactor, in accordance with the present disclosure.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “adipogenic precursor cells” or “pre-adipocytes” refer to precursor cells capable of differentiating into mature adipose cells. “Adipogenic precursor cells” or “pre-adipocytes” may be used interchangeably throughout the present disclosure. Non-limiting examples of adipogenic precursor cells include stem cells such as pluripotent stem cells (PSCs), mesenchymal stem cells (MSCs), muscle-derived stem cells (MDSCs), and adipose-derived stem cells (ADSCs) (e.g., porcine, bovine, human, avian (chicken), piscine etc.). In addition, transdifferentiated cells can also be utilized. Other adipogenic precursor cells may include, but are not limited to, dedifferentiated fat (DFAT) cells (e.g., porcine, bovine, piscine, etc.), preadipocytes (e.g., human, bovine, avian (chicken), murine, piscine, etc.), and fibroblasts (e.g., avian (chicken), bovine, porcine, murine, piscine, etc.).

As used herein, “adipose cells” are fat cells or adipocytes. “Adipose cells”, “fat cells”, and “adipocytes” may be used interchangeably throughout the present disclosure.

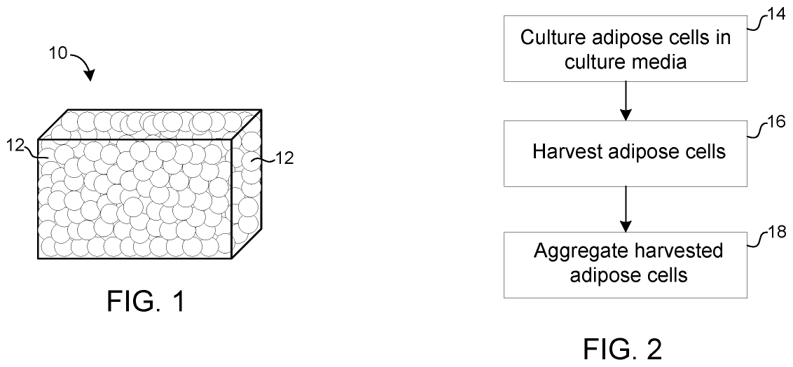

Referring now to the drawings, and with specific reference to FIG. 1, a schematic representation of cultured adipose tissue 10 is shown. The cultured adipose tissue 10 may include adipose cells 12 (or adipocytes 12) in an extracellular matrix. The cultured adipose tissue 10 may be arranged in a defined three-dimensional (3D) shape and may have a size on the macroscale (i.e., millimeter scale and greater). Although a cube-like structure is shown in FIG. 1 for simplicity, it will be understood that the cultured adipose tissue 10 may have any suitable 3D shape in practice. In some embodiments, the cultured adipose tissue 10 may be a food product suitable for consumption. In other embodiments, the cultured adipose tissue 10 may be incorporated as an ingredient in a food product suitable for consumption. As explained further below, the cultured adipose tissue 10 is produced using a method that circumvents the mass transport limitations associated with directly culturing bulk or large scale 3D tissues.

Turning to FIG. 2, a general exemplary method for producing the cultured adipose tissue 10 is shown. At a first block 14, a mass of adipose cells 12 (individual adipose cells 12, or small clusters of adipose cells 12) are cultured from adipogenic precursor cells in culture media. For instance, the block 14 may include growing adipogenic precursor cells to confluency (or to a desired coverage/number of cells on a surface or in suspension) in a first culture media, and then differentiating the adipogenic precursor cells into adipose cells 12 in a second culture media. The first culture media may be an adipogenic induction media which supports proliferation of the adipogenic precursor cells, and the second culture media may be a lipid accumulation media to provide large numbers of lipid-filled adipose cells 12. In alternative embodiments, a single culture medium may be used for both proliferation/growth of the adipogenic precursor cells and for differentiation of the adipogenic precursor cells into adipose cells. The culture time may be tuned to control lipid yield and droplet size. For example, Applicant has found that longer culture times (about a month) yield droplets comparable to in vivo fat (e.g., chicken). In some embodiments, the adipose cells 12 may be genetically modified to improve their growth and lipid accumulation for more efficient scale up.

After the adipose cells 12 have accumulated sufficient lipid and a desired amount of adipose cells 12 are generated, the culture is ended, and the lipid-laden adipose cells 12 are harvested according to a block 16. In some embodiments, the block 16 may include detaching the adipose cells 12 from a substrate, and draining the adipose cells of non-cell liquid.

At a next block 18, the harvested adipose cells 12 may be aggregated in a 3D mold (e.g., a 3D printed mold) having a desired 3D shape to generate the 3D adipose tissue 10. In some embodiments, the block 18 may involve embedding the harvested adipose cells 12 in a hydrogel or a binder in a 3D mold. Suitable hydrogels or binders include, but are not limited to, food safe compounds such as alginate, cellulose, gelatin, starch, hyaluronic acid, fibrin, carrageenan, guar gum, inulin, konjac, oat bran, pectin, locust bean gum, xanthan gum, soy protein, wheat gluten, zein protein, silk fibroin, pullulan, cellulose derivatives and combinations thereof. In some embodiments, the hydrogel or binder is alginate which is a material used as a fat replacer in the food industry. For instance, the block 18 may include mixing the harvested and drained adipose cells 12 with an alginate solution at a specified volumetric ratio in the 3D mold. In one specific embodiment, a slow gelling alginate solution may be prepared by adding calcium carbonate and glucono delta-lactone (GDL) powders to an alginate solution, and the slow gelling alginate solution may be combined with the harvested and drained adipose tissue at a 1:1 volumetric ratio in a 3D printed mold (see Example 3).

In some aspects, the block 18 may involve cross-linking the harvested adipose cells 12 in a 3D mold. The cross-linking may be carried out using a suitable protein-protein cross-linking enzyme such as, but not limited to, a transglutaminase, a tyrosinase, a peroxidase, and a laccase. In some aspects, cross-linking the harvested adipose cells includes enzymatically cross-linking the harvested adipose cells using transglutaminase. For example, cross-linking the harvested adipose cells may involve mixing a solution of transglutaminase with the harvested adipose cells at a specified volumetric ratio in a 3D mold (see Example 3). The block 18 may further include adding helper proteins during the cross-linking. In some embodiments, the helper proteins may be selected from casein and gelatin. Chemical crosslinking can also be used when the reactants or catalysts are food safe, such as EDC-NHS reactions between acid and amine groups. Photochemical crosslinking can also be utilized where photosensitizers are food safe.

Alternatively, other types of immobilization, entrapment, or crosslinking agents may be used for adipose cell aggregation such as, but not limited to, polymers functionalized with aldehyde groups, genipin, phenolic compounds, and combinations thereof. Suitable polymers functionalized with aldehyde groups include, but are not limited to, periodate oxidized pectin, dextran, chitosan, Arabic gum, sucrose, raffinose, stachyose, cyclodextrin, and starch. Suitable phenolic compounds include, but are not limited to, caffeic acid, chlorogenic acid, caftaric acid, quercetin, and rutin derived from plants such as grapes and coffee.

The adipose cells 12 or the adipose tissue 10 may be supplemented at various stages to tune the sensorial characteristics (e.g., texture, color, and flavor) and/or the nutritional attributes of the cultured adipose tissue 10. For example, supplementation with additives such as, but not limited to, flavorants, colorants, texturizers, vitamins, minerals, amino acids, proteins/peptides, and fatty acids is also encompassed by the present disclosure. In some embodiments, tunable control of fat nutrition and health may be implemented. The fatty acid composition of the cultured adipose tissue may be tailored via cell feeding strategies, such as by supplementing fatty acids into the culture media during in vitro culture. Genetic interventions may also serve to bolster the nutrition of the cultured adipose tissue 10. For example, in one embodiment, omega 3 desaturases may be expressed or pathways to produce lipophilic nutrients (e.g., beta carotene, vitamin A) may be activated in the adipose cells 12. This may be beneficial to the consumer as certain nutrients are more bioavailable when consumed in food versus a micronutrient supplement. Additionally, the texture of the cultured fat may be tunable based on variables such as the hydrogel/binder (e.g., alginate) concentration, cross-linker levels, and the inclusion of helper proteins (e.g., casein, gelatin, etc.) during cross-linking. In one embodiment, the cultured adipose cells 12 may be supplemented with methylated branched fatty acids to impart a “mutton” flavor in the cultured adipose cells 12. Additionally, the relative extracellular matrix production and fat production levels may be optimized pending the texture, taste, and/or nutritional outcomes desired.

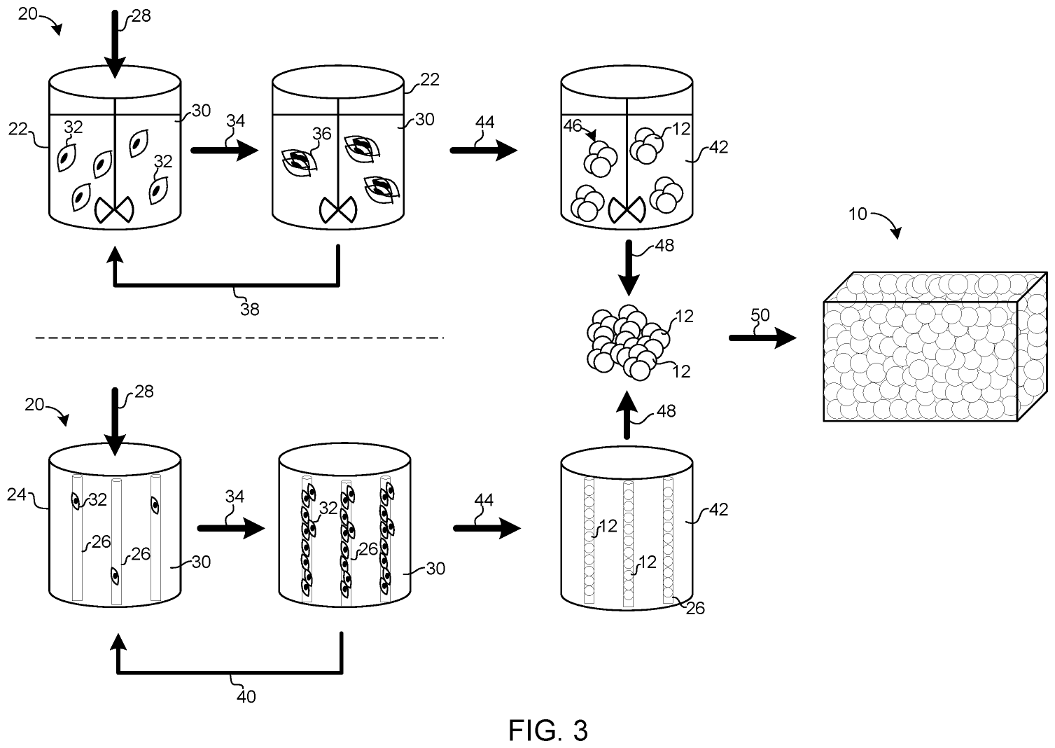

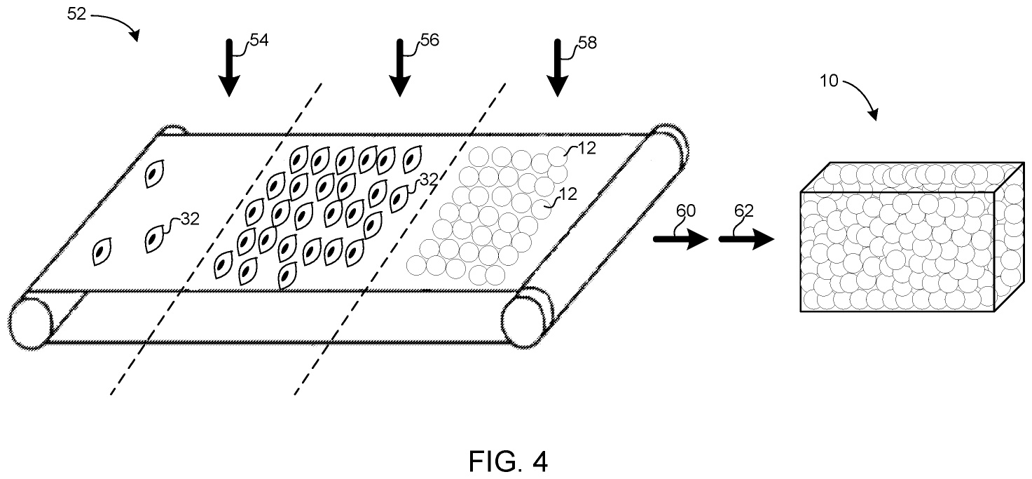

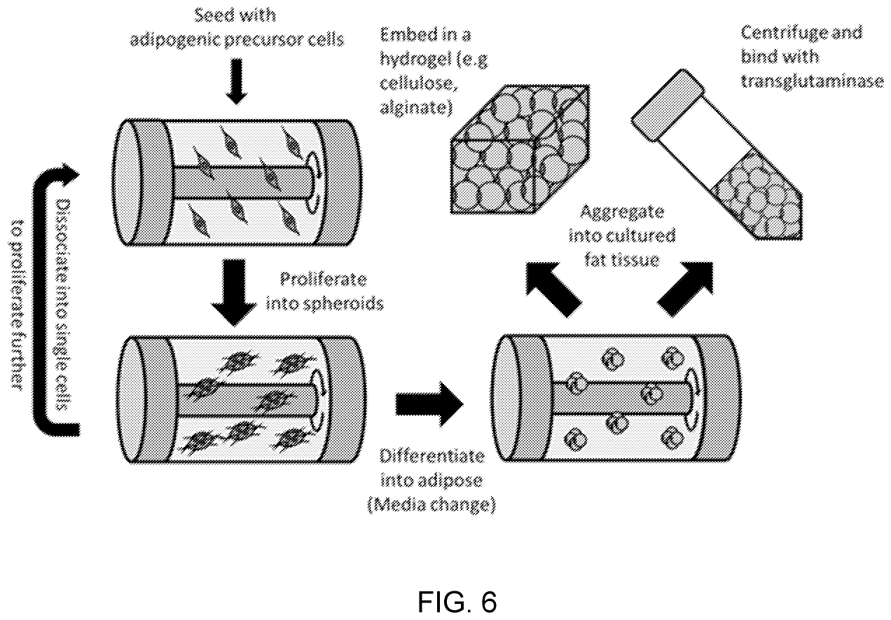

FIG. 3 shows scalable processes for the mass production of the cultured adipose tissue 10. The processes may be carried out in a bioreactor 20, such as a stirred suspension tank bioreactor 22 (top) or a hollow fiber bioreactor 24 having hollow fiber membranes 26 (bottom). Other types of bioreactors apparent to those skilled in the art may also be used and are within the scope of the present disclosure such as, but not limited to, rotating wall vessel bioreactors (RWVBs), fixed bed bioreactors, and packed bed bioreactors. Referring to FIG. 6, one exemplary arrangement using RWVBs is illustrated. Production of the adipose tissue 10 in the bioreactor 20 may involve seeding 28 a first culture media 30 (adipogenic induction media) in the bioreactor 20 with adipogenic precursor cells 32. The adipogenic precursor cells 32 may then proliferate 34 to confluency (or to a desired coverage/number of cells on a surface or in suspension) in the bioreactor 20. In some embodiments, the adipogenic precursor cells 32 may form small aggregates or spheroids 36 as they proliferate (see FIG. 3, top). The spheroids 36 may be dissociated 38 into single adipogenic precursor cells 32 and allowed to proliferate 34 further (see FIG. 3, top). In the hollow fiber reactor 24, the adipogenic precursor cells 32 may proliferate on the surface of the hollow fiber membranes 26 (see FIG. 4, bottom). In this case, the adipogenic precursor cells 32 may be detached 40 from the hollow fiber membranes 26, and the detached adipogenic precursor cells 32 may be used to re-seed the media 30 for further proliferation 34.

As the first culture media 30 is changed to a second culture media 42 (the lipid accumulation media), the cells may accumulate lipids and differentiate 44 into adipose cells 12. The adipose cells 12 may grow separately or in small clusters 46 (see FIG. 3, top). In the hollow fiber bioreactor 24, the adipose cells 12 may develop on the surface of the hollow fiber membranes 26. In some embodiments, a single culture medium may be used for both proliferation 34 and differentiation 44. After the adipose cells 12 have grown and accumulated sufficient lipid, the adipose cells 12 may be harvested 48. In the hollow fiber bioreactor 24, the harvesting may involve detaching the adipose cells 12 from the hollow fiber membranes 26. The hollow fiber membranes 26 can in some cases be edible, thereby obviating the need for detachment. The harvested adipose cells 12 may then be aggregated 50 in a 3D mold to provide the cultured adipose tissue 10. As explained above, suitable methods for binding and aggregating 50 the adipose cells 12 include cross-linking (e.g., enzymatic cross-linking with transglutaminase), as well as embedding the adipose cells 12 in hydrogels such as alginate.

The culturing process of the present disclosure may be compatible with two dimensional (2D) culture strategies. In some embodiments, the adipose cells 12 may be cultured in thin layers on a 2D substrate such as culture plates, and then aggregated into the 3D adipose tissue 10 according to the above-described procedures. For instance, the adipogenic precursor cells 32 may be grown to confluency (or to a desired coverage/number of cells on a surface or in suspension) and differentiated into the adipose cells 12 on the 2D substrate. Harvesting or collecting the adipose cells 12 from the 2D substrate followed by aggregating the harvested adipose cells 12 may provide the cultured adipose tissue 10. In some embodiments, the 2D substrate may by edible and incorporated into the final food product, such that the adipose cells 12 do not need to be detached from the 2D substrate.

In a continuous, assembly line-like process for the mass production of the cultured adipose tissue 10, the 2D substrate may be a conveyor belt 52 (see FIG. 4). The continuous production process may involve seeding 54 the adipogenic precursor cells 32 onto the conveyor belt 52 having a culture media thereon. The adipogenic precursor cells 32 may then proliferate 56 to confluency (or to a desired coverage/number of cells on a surface or in suspension) on the conveyor belt 52. Changing the culture media to lipid accumulation media may allow the adipogenic precursor cells 32 to accumulate lipid and differentiate 58 into the adipose cells 12. Alternatively, a single culture medium may be used for both proliferation 56 and differentiation 58. The adipose cells 12 may be harvested 60 by detachment from the conveyor belt 52, and then aggregated 62 according to the above-described procedures to provide the cultured adipose tissue 10.

The technology disclosed herein provides a novel and scalable approach to cultured fat generation. The present disclosure leverages large-scale cell proliferation and scale up technology to generate a required amount of in vitro adipose cells, after which the cells are aggregated or packed into a solid 3D construct on the macroscale. The adipose cells are cultured in thin layers (2D culture) or in bioreactors with easy access to the culture media, followed by aggregation into macroscale 3D tissues after sufficient adipocyte maturation. The aggregation of adipocytes or adipocyte clusters recapitulates native fat tissue from a sensory perspective as adipose tissue in vivo is largely a dense aggregation of lipid filled adipocytes with a sparse extracellular matrix. Furthermore, the compatibility of the adipose tissue production method with 2D culture strategies allows for a continuous production process with a conveyor belt assembly line approach.

Additionally, the method of the present disclosure produces bulk cultured adipose tissue in a way that circumvents the mass transport limitations associated with directly culturing or engineering large 3D tissues. Aggregation at the end of cell culture removes the need for nutrient delivery to the adipose cells via vascularization or an elaborate tissue perforation system. This is because, for food applications, the cultured adipose cells do not need to stay alive once formed into the final edible tissue. This is analogous to meat production in conventional animal agriculture where muscle and fat cells gradually cease to be viable after slaughter. In contrast, for medical applications, cells in 3D tissues may be expected to remain viable to be used for implantation into the body or for testing in an in vitro tissue model. Accordingly, the adipose tissue production method of the present disclosure is less costly than other methods that rely on complex perfusion and mixing systems to distribute nutrients during cell growth.

According to the methods of the present disclosure, monocultures of adipocytes and pre-adipocytes may be sufficient for the production of large fat droplets without the need for supporting cell types. Standard cell culture conditions are sufficient for the type of adipocyte culture outlined in this disclosure, and no specific coatings on tissue culture plastics were required to achieve desired adipocyte growth and development. Furthermore, the pre-adipocytes and adipocytes of various livestock species may be grown in serum-free culture media according to the present disclosure, thereby eliminating a major obstacle in in vitro fat culture. These advantages further help reduce production costs. Co-cultures can also be considered for enhanced fat outcomes, such as the use of fibroblasts or muscle cells in the cultures, such as to increase the quality of the fat products or to alter the texture and composition.

Applicant has also observed that a large subpopulation of the cultured adipocytes adhere strongly to tissue culture plates and do not float away, avoiding issues of lift-off of adherent adipocytes in vitro due to increasing buoyancy as the adipocytes become fatty. The 2D culture systems disclosed herein self-sort for adherent cell populations.

### EXAMPLES

### Example 1: Timeline of 3T3-L1 Adipogenic Differentiation

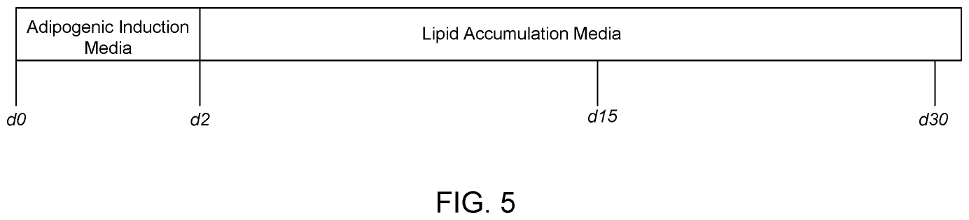

FIG. 5 shows a timeline for differentiation of 3T3-L1 adipogenic cells. Days 0 (d0), 2 (d2), 15 (d15), and 30 (d30) are indicated on the timeline. Confluent pre-adipocytes were grown in adipogenic induction media for the first two days and then switched to lipid accumulation media until harvest for cultured fat tissue formation on day 15 (see Example 2). Additional samples were grown in lipid accumulation media for 30 days to analyze lipid accumulation over longer-term culture.

### Example 2: Harvest of Lipid-Laden Adipocytes and Formation of 3D Cultured Fat Constructs

Following lipid accumulation, lipid-filled adiopocytes were detached using a cell scraper. The adipocytes were then drained of non-cell liquid using a 0.22 micrometer vacuum filter. After detaching and draining (it should be noted that it is possible to drain the liquid from the cells prior to detachment, so that once detached, the result is a raw adipocyte slurry), the in vitro adipocytes were then combined with transglutaminase or alginate and formed into discrete macroscale tissues in a 3D printed mold. Finally, 3D cultured fat constructs were mechanically tested for compressive strength, fluorescently stained for lipid and analyzed for volatile compounds.

### Example 3: Methods for Generating 3D Cultured Fat Using Alginate or Transglutaminase

Alginate aggregation. Slow-gelling alginate solution was prepared by adding calcium carbonate and glucono delta-lactone (GDL) powders to a 1.6% or a 3.2% alginate solution before combining with the harvested and drained in vitro adipose tissue at a 1:1 volumetric ratio in a 3D printed mold.

Transglutaminase aggregation. Cultured fat was produced by mixing a 15% solution of transglutaminase with drained adipose tissue at a 2:8 volumetric ratio in a 3D printed mold.

What is claimed:

1. A method for producing cultured adipose tissue, comprising:
growing adipogenic precursor cells in a first culture media;
differentiating the adipogenic precursor cells to adipose cells in a second culture media;
harvesting the adipose cells; and
aggregating the harvested adipose cells to provide the cultured adipose tissue.

2. The method of claim 1, wherein growing the adipogenic precursor cells in the first culture media comprises seeding the adipogenic precursor cells into a bioreactor containing the first culture media, and allowing the adipogenic precursor cells to proliferate in the bioreactor.

3. The method of claim 2, wherein differentiating the adipogenic precursor cells to adipose cells comprises changing the first culture media to the second culture media in the bioreactor.

4. A method for producing cultured adipose tissue, comprising:
growing adipogenic precursor cells in a culture media;
differentiating the adipogenic precursor cells to adipose cells in the culture media;
harvesting the adipose cells; and
aggregating the harvested adipose cells to provide the cultured adipose tissue.

5. The method of claim 1 or 4, wherein the method is carried out in a bioreactor.

6. The method of claim 2, 3 or 5, wherein the bioreactor is a stirred suspension tank bioreactor.

7. The method of claim 2, 3 or 5, wherein the bioreactor is a rotating wall vessel bioreactor.

8. The method of claim 2, 3 or 5, wherein the bioreactor is a hollow fiber bioreactor.

9. A method for producing cultured adipose tissue, comprising:
growing adipogenic precursor cells on a two-dimensional (2D) substrate;
differentiating the adipogenic precursor cells to adipose cells on the 2D substrate;
harvesting the adipose cells; and
aggregating the harvested adipose cells to provide the cultured adipose tissue.

10. The method of claim 9, wherein growing the adipogenic precursor cells on the 2D substrate comprises seeding the adipogenic precursor cells onto the 2D substrate, and allowing the adipogenic precursor cells to proliferate on the 2D substrate.

11. The method of claim 9 or 10, wherein the 2D substrate forms at least a portion of a conveyor belt.

12. The method of any of claims 9 to 11, wherein the method is performed continuously in an assembly line process.

13. A method for producing cultured adipose tissue, comprising:
culturing adipose cells from adipogenic precursor cells in culture media;
harvesting the adipose cells after a desired amount of adipose cells are produced; and
aggregating the harvested adipose cells to provide the cultured adipose tissue.

14. The method of any one of the preceding claims, wherein the adipogenic precursor cells are pluripotent stem cells.

15. The methods of any of claims 1 to 13, wherein the adipogenic precursor cells are mesenchymal stem cells.

16. The method of any one of the preceding claims, wherein aggregating the harvested adipose cells comprises mixing the harvested adipose cells with a hydrogel or binder in a 3D mold.

17. The method of claim 16, wherein the hydrogel or binder is selected from the group consisting of alginate, cellulose, gelatin, starch, hyaluronic acid, fibrin, carrageenan, guar gum, inulin, konjac, oat bran, pectin, locust bean gum, xanthan gum, soy protein, wheat gluten, zein protein, silk protein, cellulose derivatives, pullulan and combinations thereof.

18. The method of any one of the preceding claims, wherein aggregating the harvested adipose cells comprises mixing the harvested adipose cells with alginate.

19. The method of claim 18, wherein mixing the harvested adipose cells with alginate comprises:
adding calcium carbonate and glucono delta-lactone to a solution of the alginate; and
combining the harvested adipose cells with the solution of the alginate in the 3D mold.

20. The method of any of claims 1 to 15, wherein aggregating the harvested adipose cells comprises cross-linking the harvested adipose cells in a 3D mold.

21. The method of claim 20, wherein cross-linking the harvested adipose cells comprises cross-linking the harvested adipose cells using an enzyme selected from the group consisting of a transglutaminase, a tyrosinase, a peroxidase, and a laccase.

22. The method of claim 20, wherein cross-linking the harvested adipose cells in the 3D mold comprises enzymatically cross-linking the harvested adipose cells using transglutaminase.

23. The method of claim 22, wherein cross-linking the harvested adipose cells with transglutaminase comprises mixing a solution of transglutaminase with the harvested adipose cells at a predetermined volumetric ratio in the 3D mold.

24. The method of claim 20, wherein cross-linking the harvested adipose cells comprises cross-linking the harvested adipose cells using a cross-linker selected from the group consisting of polymers functionalized with aldehyde groups, genipin, and phenolic compounds.

25. The method of claim 24, wherein the polymers functionalized with aldehyde groups are selected from a group consisting of periodate oxidized pectin, dextran, chitosan, Arabic gum, sucrose, raffinose, stachyose, cyclodextrin, and starch.

26. The method of claim 24, wherein the phenolic compound is selected from the group consisting of caffeic acid, chlorogenic acid, caftaric acid, quercetin, and rutin.

27. The method of any one of the preceding claims, wherein aggregating the harvested adipose cells comprises adding a protein during the aggregation.

28. The method of claim 27, wherein the protein is selected from casein and gelatin.

29. The method of any one of the preceding claims, further comprising draining the adipose cells to remove non-cell liquid after harvesting the adipose cells and prior to aggregating the harvested adipose cells.

30. The method of any one of the preceding claims, wherein the cultured adipose tissue has a size on the macroscale.

31. The method of any one of the preceding claims, wherein the cultured adipose tissue has a defined 3D shape.

32. The method of any one of the preceding claims, further comprising supplementing the adipose cells with methylated branched fatty acids.

33. The method of any one of the preceding claims, wherein the adipose cells express omega 3 desaturases.

34. Cultured adipose tissue comprising adipose cells embedded in a hydrogel or binder, wherein the cultured adipose tissue has a three-dimensional (3D) shape and a size on the macroscale.

35. The cultured adipose tissue of claim 34, wherein the hydrogel or binder is selected from the group consisting of alginate, cellulose, gelatin, starch, hyaluronic acid, fibrin, carrageenan, cellulose, guar gum, inulin, konjac, oat bran, pectin, locust bean gum, xanthan gum, soy protein, wheat gluten, zein protein, and combinations thereof.

36. The cultured adipose tissue of claim 34 or 35, wherein the mass of adipose cells are cross-linked with alginate.

37. Cultured adipose tissue comprising adipose cells cross-linked together, wherein the cultured adipose tissue has a three-dimensional (3D) shape and a size on the macroscale.

38. The cultured adipose tissue of claim 37, wherein the adipose cells are cross-linked using an enzyme selected from the group consisting of a transglutaminase, a tyrosinase, a peroxidase, and a laccase.

39. The cultured adipose tissue of claim 37, wherein the adipose cells are cross-linked with transglutaminase.

40. The cultured adipose tissue of claim 37, wherein the adipose cells are cross-linked with a cross-linker selected from the group consisting of polymers functionalized with aldehyde groups, genipin, and phenolic compounds.

41. The cultured adipose tissue of claim 40, wherein the polymers functionalized with aldehyde groups are selected from a group consisting of periodate oxidized pectin, dextran, chitosan, Arabic gum, sucrose, raffinose, stachyose, cyclodextrin, and starch.

42. The cultured adipose tissue of claim 40, wherein the phenolic compounds are selected from the group consisting of caffeic acid, chlorogenic acid, caftaric acid, quercetin, and rutin.

43. The method or cultured adipose tissue of any one of the preceding claims, wherein the cultured adipose tissue is a food product.

44. The method or cultured adipose tissue of any one of the preceding claims, wherein the cultured adipose tissue is an ingredient of a food product.

45. The method or cultured adipose tissue of any one of the preceding claims, wherein one or more components of the adipose tissue are ingredients in a food product.

46. The method or cultured adipose tissue of any one of the preceding claims, wherein the cultured adipose tissue is produced without vascularization or perfusion.
